(19) United States
(12) Patent Application Publication
(10) Pub. No.: US 2022/0409609 A1
(43) Pub. Date: Dec. 29, 2022

(54) INJECTABLE FORMULATION OF HYDROXYCHLOROQUINE

(71) Applicant: Luckwel Pharmaceuticals Inc., Cambridge, MA (US)

(72) Inventor: Kingrich Lee, Cambridge, MA (US)

(21) Appl. No.: 17/842,869

(22) Filed: Jun. 17, 2022

Related U.S. Application Data
(60) Provisional application No. 63/212,439, filed on Jun. 18, 2021.

(51) Int. Cl. A61K 31/4706; A61K 47/26; A61K 47/02; A61K 9/00

(52) U.S. Cl. CPC A61K 31/4706; A61K 47/26; A61K 47/02; A61K 9/0019

(57) ABSTRACT

The present subject matter relates to stable, low viscosity, high concentration, injectable liquid pharmaceutical compositions of hydroxychloroquine sulfate. Also described are methods of use of the compositions for the treatment rheumatoid arthritis and lupus erythematosus. A process for preparing the compositions is also described.

### CROSS-REFERENCE TO RELATED APPLICATIONS

This application claims the benefit of priority to U.S. Provisional Patent Application No. 63/212,439, filed Jun. 18, 2021, which is hereby incorporated by reference in its entirety.

### TECHNICAL FIELD

The present subject matter relates to stable, low viscosity, high concentration liquid pharmaceutical compositions of hydroxychloroquine sulfate. Also described are methods of use of the compositions for the treatment of diseases, disorders or conditions that are therapeutically responsive to hydroxychloroquine sulfate. A process for preparing the compositions is also described.

### BACKGROUND

Hydroxychloroquine sulfate is a drug for the treatment of malaria, rheumatoid arthritis, and systemic lupus erythematosus. The U.S. Food and Drug Administration (FDA) approved it 1955. Hydroxychloroquine concentrations peak at 3-4 hours after hydroxychloroquine dosing.

Hydroxychloroquine sulfate (or hydroxychloroquine) refers to 2-[[4-[(7-Chloro-4-quinolyl)amino]pentyl]ethylamino] ethanol sulfate (1:1), which was described as a racemate in U.S. Pat. No. 2,546,658, and has the below structure.

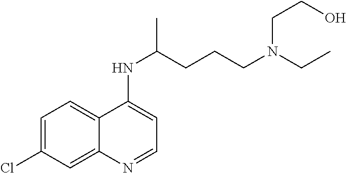 [SMILES: CCN(CCO)CCCC(C)Nc1ccnc2cc(Cl)ccc12]

The (S)-(+)-enantiomer showing lower risk profile was patented in U.S. Pat. No. 5,314,894. Solid tablets are currently available under multiple trademarks, e.g., PLAQUENIL® (NDA N009768, CONCORDIA PHARMACEUTICALS INC., approval date prior to Jan. 1, 1982; package insert available at https://dailymed.nlm.nih.gov/dailymed/drugInfo.cfm?setid=eb26c050-fc41-4183-9f8c-86351335fa64&audience=consumer). Tablets come in doses of 200 mg hydroxychloroquine sulfate and the following excipients (inactive ingredients): dibasic calcium phosphate, Hypromellose, magnesium stearate, polyethylene glycol 400, polysorbate 80, corn starch, titanium dioxide, carnauba wax, shellac, and black iron oxide.

Hydroxychloroquine sulfate is a white solid which is freely soluble in water at 25° C. Hydroxychloroquine has been found to be stable in a wide range of pharmaceutic& formulations and is a known disease-modifying antirheumatic drug (DMARD). Although the mechanism of action has yet to be elucidated, hydroxychloroquine is currently prescribed to treat rheumatoid arthritis alone, or in combination (e.g., with methotrexate and sulfasalazine).

Hydroxychloroquine sulfate is administered at levels such as 200 or 400 mg daily for the treatment of lupus erythematosus, or 400 or 600 mg daily for the treatment of rheumatoid arthritis. It may require weeks or months to achieve maximum therapeutic effect. There are currently no liquid formulations of hydroxychloroquine listed with the FDA Orange Book.

Not all patients are easily treated with solid formulations, however. Patients afflicted with dysphagia, as well as young and elderly patients, experience difficulty in swallowing solid oral dosage forms. Difficulty in swallowing leads to poor patient compliance. In addition to difficulty in successful administration, orally administered solid dosage forms are more likely to require larger doses that necessary due to inadequate absorption and may cause gastrointestinal side effects.

It would be beneficial to provide a stable, low viscosity, high concentration, injectable liquid pharmaceutical composition comprising hydroxychloroquine sulfate; however, no such suitable dosage form containing hydroxychloroquine sulfate has been disclosed in the art.

### BRIEF SUMMARY

In a first aspect, the invention provides a stable, low viscosity, liquid pharmaceutical composition comprising at least 150 mg/ml of hydroxychloroquine sulfate.

In some embodiments, the pharmaceutical composition comprises at least 180 mg/ml of hydroxychloroquine sulfate.

In some embodiments, the pharmaceutical composition further comprises a non-ionic surfactant. In one example, the non-ionic surfactant has a concentration of 0.04 mg/l. As a non-limiting example, the non-ionic surfactant can be polysorbate 20 or 80.

In some embodiments, the pharmaceutical composition further comprises an inorganic salt. As a non-limiting example, the inorganic salt can comprise sodium chloride.

In some embodiments, the pharmaceutical composition has a pH of 5.8-6.2

In some embodiments, the pharmaceutical composition alleviates at least one symptom of rheumatoid arthritis or lupus erythematosus.

In some embodiments, the pharmaceutical composition is suitable for administration by at least one of the following routes: parenteral, subcutaneous, intramuscular, intravenous, intraarticular, intrabronchial, intraabdominal, intracapsular, intracartileginous, intracavitary, intracelial, intracerebellar, intracerebroventicular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathorachic, intraunterine, intravesical, bolud, vaginal, rectal, buccal, sublingual, intranasal, and transdermal. As a preferred example, the composition is suitable for subcutaneous administration.

In another aspect, the present disclosure provides for a prefilled syringe containing the pharmaceutical compositions described herein. In one example, the prefilled syringe comprises 0.9 ml of the pharmaceutical composition.

In addition to the exemplary aspects and embodiments described above, further aspects and embodiments will become apparent by reference to the drawings and by study of the following descriptions.

Additional embodiments of the present devices, methods, kits and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment described herein.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to an “excipient” includes a single excipient as well as two or more of the same or different excipients, and the like.

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. In some embodiments, “about” means that the item, parameter or term so qualified encompasses a range of plus or minus ten percent above and/or below the value of the stated item, parameter or term.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Therapeutically effective amount” as applied to the biologically active ingredient means that amount of the active ingredient which is generally sufficient to effect a desired change in the subject. For example, where the desired effect is a reduction in an autoimmune disorder symptom, an effective amount of the active ingredient is that amount which causes at least a substantial reduction of the autoimmune disorder symptom, and without resulting in significant toxicity.

“Subject” or “patient” means a human or non-human subject receiving medical or veterinary care. Accordingly, the method as disclosed herein can be used in treating any animal, such as, for example, mammals, or the like.

“Treating” means to alleviate (or to eliminate) at least one symptom of a condition or disorder, such as, for example, cough, fever, shortness of breath, or the like, either temporarily or permanently.

### II. Hydroxychloroquine Sulfate Dosage Form

As used herein and unless otherwise specified, the term hydroxychloroquine sulfate refers to the drug in underivatized as 2-[[4-[(7-Chloro-4-quinolyl)amino]pentyl]ethylamino] ethanol sulfate (1:1) described in U.S. Pat. No. 2,546,658 or derivatized form.

The injectable pharmaceutical compositions of the present technology contain hydroxychloroquine sulfate in a concentration of between 150 and 200 mg/ml, such as between 150 and 160 mg/ml, between 160 and 170 mg/ml, between 170 and 180 mg/ml, between 180 and 190 mg/ml, or between 190 and 200 mg/ml. In addition to the hydroxychloroquine sulfate, the compositions comprise excipients compatible with an injectable solution, e.g., surfactants, inorganic salts, buffers, diluents, solubilizing agents, isotonizing agents, excipients, pH-modifiers, soothing agents, buffers, sulfur-containing reducing agents, antioxidants etc. Injectable solution may be stored in vial form. Vials of hydroxychloroquine sulfate are preferrably stored at between 4 and 20 ml of 20 mg/ml.

Any surfactant compatible with injectable solutions may be used with the present technology. In preferred embodiments, the surfactants are non-ionic surfactants. Preferred non-ionic surfactants include polysorbate 20, 40, 60, or 80, or poloxamer 188. Non-ionic surfactant concentrations may be in the range of between 0.002 to 0.006% (w/v), for example between 0.002 and 0.003% (w/v), between 0.003 and 0.005% (w/v), or between 0.005% and 0.006% (w/v).

Buffers known in the art that are compatible with injectable pharmaceutical compositions are contemplated by the present technology. Inorganic salt buffers and amino acid buffers are non-limiting examples of acceptable buffers. In one embodiment the amino acid buffer comprises histidine or a histidine derivative. In a preferred embodiment the histidine buffer is histidine hydrochloride/L-histidine. Exemplary buffer concentrations may be between 50 and 70 mM, such as between 50 and 55 mM, between 55 and 60 mM, between 60 and 65 mM, or between 65 and 70 mM; or between 25 mM and 35 mM, such as between 25 and 27 mM, between 27 and 29 mM, between 29 and 31 mM, between 31 and 33 mM, or between 33 and 35 mM; or between 5 mM and 10 mM, such as between 5 and 6 mM, between 6 and 7 mM, between 7 and 8 mM, between 8 and 9 mM, or between 9 and 10 mM. The buffer concentration is preferably sufficient to maintain a pH of between 5.0 and 7.0, such as between 5 and 5.5, between 5.5 and 6.0, between 6.0 and 6.5, or between 6.5 and 7.0.

Inorganic salts known in the art that are compatible with injectable pharmaceutical compositions are also contemplated by the present technology. Some exemplary salts include sodium chloride, potassium chloride, calcium chloride, sodium phosphate, potassium phosphate, and sodium bicarbonate. Preferred embodiments employ sodium chloride. In embodiments, concentrations of inorganic salts may be between 10 mM and 30 mM, e.g., between 10 and 15 mM, between 15 and 20 mM, between 20 and 25 mM, or between 25 and 30 mM.

The combination of the described excipients preferably yields a composition with a viscosity less than 20 cP, e.g., less than 18 cP, less than 15 cP, less than 13 cP, less than 10 cP, less than 8 cP, less than 5 cP, less than 3 cP, or less than 1 cP, at about 25° C.

### III. Methods of Administration

The pharmaceutical compositions of the present technology are administered to subjects in need thereof as needed. Preferred regimens include once daily, twice weekly, once weekly, twice monthly, or once monthly. Injections may be administered with a prefilled syringe, optionally by the subject in need thereof. Acceptable injection routes include parenteral, subcutaneous, intramuscular, intravenous, intraarticular, intrabronchial, intraabdominal, intracapsular, intracartileginous, intracavitary, intracelial, intracerebellar, intracerebroventicular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathorachic, intraunterine, intravesical, bolud, vaginal, rectal, buccal, sublingual, intranasal, and transdermal. Preferably the route of injection is subcutaneous.

Administration of the present pharmaceutical compositions may be used as necessary to alleviate symptoms of diseases or disorders that respond to hydroxychloroquine sulfate. Preferred diseases and disorders include rheumatoid arthritis and lupus erythematosus. Administration may be repeated multiple times to achieve the desired maximum effect, for example between 1 and 10 times. Alternatively, an administration regimen may be carried out between one day and one month to achieve the desired maximum effect. Administration may then be carried out as needed to relieve the symptoms.

Administration of the present pharmaceutical compositions may be concurrent with a second medication. The second mediation may target the same disease or disorder as the present compositions, or a different diseases or disorders.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A stable, low viscosity, liquid pharmaceutical composition comprising at least 150 mg/ml of hydroxychloroquine sulfate.

2. The pharmaceutical composition of claim 1, comprising at least 162 mg/0.9 ml or 180 mg/ml of hydroxychloroquine sulfate.

3. The pharmaceutical composition of claim 1 or claim 2, further comprising a non-ionic surfactant.

4. The pharmaceutical composition of claim 3, wherein the non-ionic surfactant has a concentration of 0.04 mg/l.

5. The pharmaceutical composition of claim 3 or claim 4, wherein the non-ionic surfactant is polysorbate 20 or 80.

6. The pharmaceutical composition of any one of claims 1-5, further comprising an inorganic salt.

7. The pharmaceutical composition of claim 6, wherein the inorganic salt comprises sodium chloride.

8. The pharmaceutical composition of any one of claims 1-7, wherein the composition has a pH of 5.8-6.2

9. The pharmaceutical composition of any one of claims 1-8, wherein the composition alleviates at least one symptom of rheumatoid arthritis or lupus erythematosus.

10. The pharmaceutical composition of any one of claim 1-9, wherein the composition is suitable for administration by at least one of the following routes: parenteral, subcutaneous, intramuscular, intravenous, intraarticular, intrabronchial, intraabdominal, intracapsular, intracartileginous, intracavitary, intracelial, intracerebellar, intracerebroventicular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathorachic, intraunterine, intravesical, bolud, vaginal, rectal, buccal, sublingual, intranasal, and transdermal.

11. The pharmaceutical composition of claim 10, wherein the composition is suitable for subcutaneous administration.

12. A prefilled syringe containing the pharmaceutical composition of any one of claims 1-11.

13. The prefilled syringe of claim 12 comprising 0.9 ml of the pharmaceutical composition.

14. A method for treating rheumatoid arthritis or lupus erythematosus, comprising administering the pharmaceutical composition of any one of claims 10-13.
